(19) United States
(12) Patent Application Publication
(10) Pub. No.: US 2021/0196232 A1
(43) Pub. Date: Jul. 1, 2021

(54) ULTRASOUND LENS MODULE AND ULTRASOUND NEEDLE DEVICE

(71) Applicant: METAL INDUSTRIES RESEARCH & DEVELOPMENT CENTRE, Kaohsiung (TW)

(72) Inventors: Yu-Lin SONG, New Taipei City (TW); Shih-Hua HUANG, Kaohsiung City (TW); I-Chun CHUNG, KAOHSIUNG CITY (TW); Yen-Jie HUANG, Kaohsiung City (TW)

(21) Appl. No.: 16/726,839

(22) Filed: Dec. 25, 2019

(51) Int. Cl. A61B 8/00; G10K 11/30; A61B 8/08

(52) U.S. Cl. CPC A61B 8/4488; A61B 8/0841; G10K 11/30

(57) ABSTRACT

An ultrasound lens module and an ultrasound needle device are provided. The ultrasound needle device includes a transducer layer and the ultrasound lens module. The ultrasound lens module is disposed on the transducer layer to focus the ultrasound emitted from the transducer layer, in which the ultrasound lens module includes a metamaterial layer. The metamaterial layer includes a flat portion and a micro structure portion. The micro structure portion is located on a surface of the flat portion, in which the micro structure portion includes at least one intrusion structure. When the number of the at least one intrusion structure is one, the intrusion structure is disposed on the flat portion in a spiral shape. When the number of the at least one intrusion structure is greater than one, the intrusion structures are disposed on the flat portion in spiral shapes or in a shape of concentric circles.

### BACKGROUND

### Field of Invention

The present invention relates to an ultrasound lens module and an ultrasound needle device.

### Description of Related Art

Epidural anesthesia is an anesthesia method for injecting a local anesthetic into the epidural space to perform a reversible blocking in the spinal nerve. Nowadays, positioning an epidural needle in clinics relies on anesthesiologist's experience. However, long-term anesthesia experience is required for positioning the epidural needle. In the current industry, an ultrasound needle device is developed for positioning the epidural needle, in which the ultrasound needle device senses the positon of the epidural needle by using an ultrasound wave. However, the conventional ultrasound needle device cannot meet user's demands.

### SUMMARY

Embodiments of the present invention provide an ultrasound lens module and an ultrasound needle device capable of improving the directivity of the ultrasound wave, thereby increasing the sensing distance of the ultrasound needle device and the resolution of an ultrasound image.

In accordance with an embodiment of the present invention, the ultrasound lens module includes a metamaterial layer. The metamaterial layer includes a flat portion and a microstructure portion. The microstructure portion is located on a surface of the flat portion, in which the microstructure portion includes at least one intrusion structure. When a number of the at least one intrusion structure is one, the intrusion structure is disposed on the flat portion in a spiral shape. When the number of the at least one intrusion structure is greater than one, the intrusion structures are disposed on the flat portion in spiral shapes or in a shape of concentric circles.

In some embodiments, a pitch of the at least one intrusion structure is equal to a multiple of a half of a wavelength of the ultrasound wave or to a multiple of a quarter of the wavelength of the ultrasound wave.

In some embodiments, a height of the at least one intrusion structure is equal to a multiple of a half of a wavelength of the ultrasound wave or to a multiple of a quarter of the wavelength of the ultrasound wave.

In some embodiments, an angle included between the at least one intrusion structure and the surface of the flat portion is greater than 90 degrees and smaller than 180 degrees.

In some embodiments, the material of the metamaterial layer is ceramic or polymethyl methacrylate (PMMA).

In accordance with an embodiment of the present invention, the ultrasound needle device includes a transducer layer and an ultrasound lens module. The transducer layer is configured to emit an ultrasound wave. The ultrasound lens module is disposed on the transducer layer to focus the ultrasound wave. The ultrasound lens module includes a metamaterial layer. The metamaterial layer includes a flat portion and a microstructure portion. The microstructure portion is located on a surface of the flat portion, in which the microstructure portion includes at least one intrusion structure. When a number of the at least one intrusion structure is one, the intrusion structure is disposed on the flat portion in a spiral shape. When the number of the at least one intrusion structure is greater than one, the intrusion structures are disposed on the flat portion in spiral shapes or in a shape of concentric circles.

In some embodiments, a pitch of the at least one intrusion structure is equal to a multiple of a half of a wavelength of the ultrasound wave or to a multiple of a quarter of the wavelength of the ultrasound wave.

In some embodiments, a height of the at least one intrusion structure is equal to a multiple of a half of a wavelength of the ultrasound wave or to a multiple of a quarter of the wavelength of the ultrasound wave.

In some embodiments, an angle included between the at least one intrusion structure and the surface of the flat portion is greater than 90 degrees and smaller than 180 degrees.

In some embodiments, the material of the metamaterial layer is ceramic or polymethyl methacrylate (PMMA).

### BRIEF DESCRIPTION OF THE DRAWINGS

The invention can be more fully understood by reading the following detailed description of the embodiment, with reference made to the accompanying drawings as follows.

FIG. 1 is a schematic diagram illustrating a structure of an ultrasound needle device in accordance with embodiments of the present invention.

FIG. 2 is a schematic diagram illustrating a top view of an ultrasound sensor in accordance with embodiments of the present invention.

FIG. 3 is a schematic diagram illustrating a cross-sectional view of the ultrasound sensor along a cut line A-A′.

FIG. 4 is a schematic diagram illustrating a top view of an ultrasound sensor in accordance with embodiments of the present invention.

FIG. 5 is a schematic diagram illustrating a cross-sectional view of the ultrasound sensor along a cut line B-B′.

FIG. 6 is a schematic diagram illustrating a top view of an ultrasound sensor in accordance with embodiments of the present invention.

FIG. 7 is a schematic diagram illustrating a cross-sectional view of the ultrasound sensor along a cut line C-C′.

### DETAILED DESCRIPTION

FIG. 1 is a schematic diagram illustrating a structure of an ultrasound needle device 100 in accordance with embodiments of the present invention. The ultrasound needle device 100 includes a cylinder 110 and a connection body 120. An ultrasound sensor (not shown) is disposed on a terminal 112 of the cylinder 110 to emit/receive an ultrasound wave. The connection body 120 is configured to be connected to an ultrasound image system to enable electric signals of the ultrasound sensor to be transmitted to the ultrasound image system via the connection body 120.

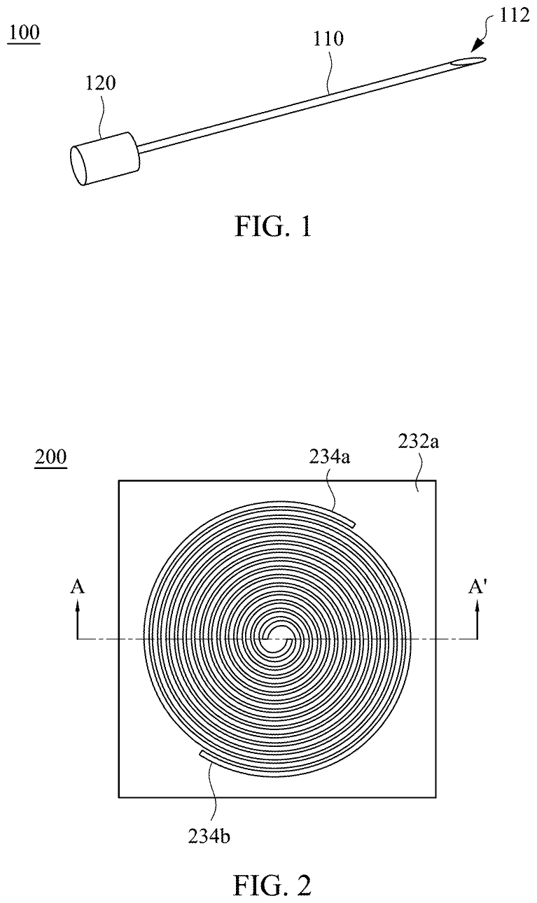

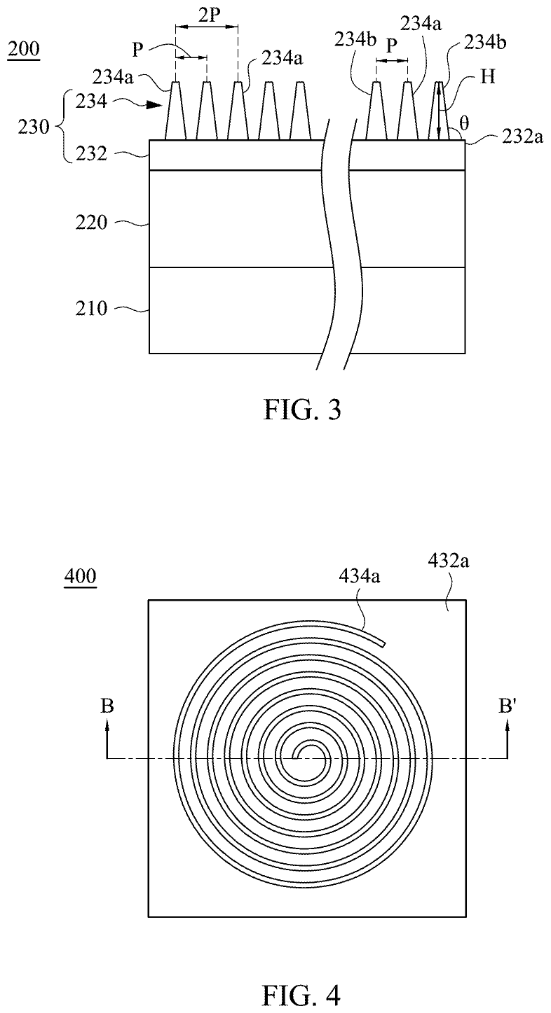

Referring to FIG. 2 and FIG. 3 simultaneously, FIG. 2 is a schematic diagram illustrating a top view of an ultrasound sensor 200 in accordance with embodiments of the present invention, and FIG. 3 is a schematic diagram illustrating a cross-sectional view of the ultrasound sensor 200 along a cut line A-A′. The ultrasound sensor 200 includes a fixing layer 210, a transducer layer 220 and a metamaterial layer 230, in which the transducer layer 220 and the metamaterial layer 230 are disposed on the fixing layer 210 sequentially. The transducer layer 220 is configured to emit the ultrasound wave. In one embodiment of the present invention, the transducer layer 220 is a piezoelectric material layer, such as a layer of ceramic material. However, embodiments of the present invention are not limited thereto. In other embodiments of the present invention, the transducer layer 220 can be a layer of capacitive micromachined ultrasonic transducer (CMUT) or a layer of piezoelectric micromachined ultrasonic transducers (PMUT). In addition, in one embodiment of the present invention, the ultrasound wave has a frequency of at least 20 MHz. In some embodiments, the frequency of the ultrasound wave is in a range from 20 MHz to 40 MHz.

In order to enable the ultrasound wave emitted from the transducer layer 220 to have a higher directivity, embodiments of the present invention provide a ultrasound lens module to focus the ultrasound wave emitted from the transducer layer 220. In this embodiment, the ultrasound lens module includes a metamaterial layer 230 disposed on the transducer layer 220 to focus the ultrasound wave emitted from the transducer layer 220. As shown in FIG. 2, the metamaterial layer 230 includes a flat portion 232 and a microstructure portion 234, in which the flat portion 232 is located between the microstructure portion 234 and the transducer layer 220 and has a flat surface 232a. The microstructure portion 234 is located on the surface 232a of the flat portion 232 and includes at least one intrusion structure, for example intrusion structures 234a and 234b. In this embodiment, the intrusion structures 234a and 234b are respectively arranged on the surface 232a of the flat portion 232 in a spiral shape, as shown in FIG. 2. In FIG. 2, the intrusion structures 234a and 234b are extended in spiral shapes and parallel to each other.

As shown in FIG. 3, a cross-sectional view of each of the intrusion structures 234a and 234b is in a taper shape, and there is an angle θ included between the surface 232a of the flat portion 232 and a surface of the intrusion structure 234a/234b. In this embodiment, the angle θ is greater than 90 degrees and smaller than 180 degrees. There is a pitch P between the two intrusion structures 234a and 234b, and each of the intrusion structures 234a and 234b is formed on the flat portion 232 to have a pitch 2P. In this embodiment, the pitch P is equal to a multiple of a half of a wavelength of the ultrasound wave, or to a multiple of a quarter of the wavelength of the ultrasound wave. Each of the intrusion structures 234a and 234b has a height H, and the height H is equal to a multiple of a half of a wavelength of the ultrasound wave or to a multiple of a quarter of the wavelength of the ultrasound wave.

It can be understood from the above illustration that the metamaterial layer 230 of this embodiment focus the ultrasound wave emitted from the transducer layer 220 through the intrusion structures 234a and 234b formed in spiral shapes, thereby enabling the ultrasound wave emitted from the transducer layer 220 to have a higher directivity, and the sensing distance of the ultrasound needle device 100 and the resolution of the ultrasound image of the ultrasound image system are increased accordingly.

In addition, a number of the intrusion structures of the metamaterial layer is not limited to the above embodiments, and the shapes and arrangement of the intrusion structures are not limited to the above embodiments. For example, in another one embodiment of the present invention, the metamaterial layer includes only one intrusion structure. For another example, in further another one embodiment of the present invention, the intrusion structures have annular shapes and arranged in a shape of concentric circles.

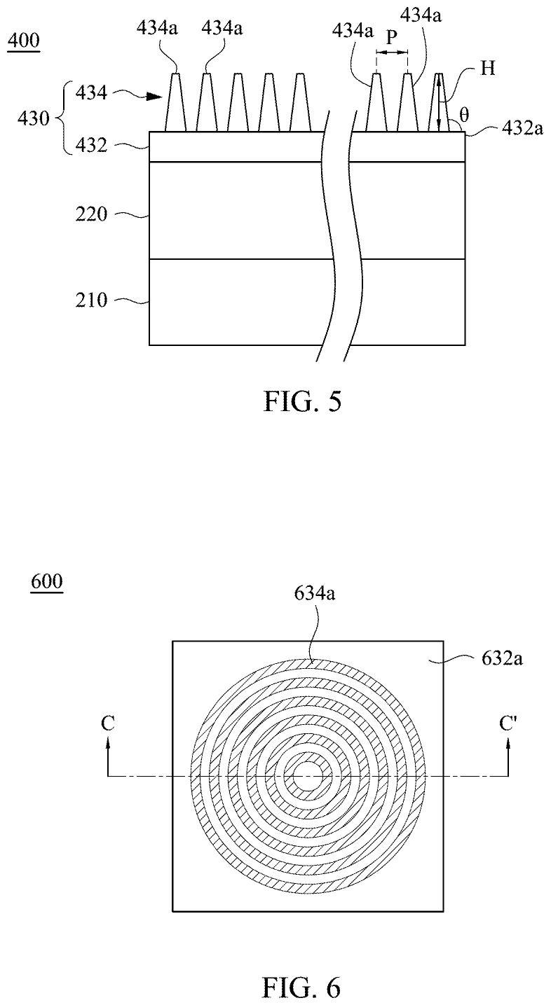

Referring to FIG. 4 and FIG. 5 simultaneously, FIG. 4 is a schematic diagram illustrating a top view of an ultrasound sensor 400 in accordance with embodiments of the present invention, and FIG. 5 is a schematic diagram illustrating a cross-sectional view of the ultrasound sensor 400 along a cut line B-B′. The ultrasound sensor 400 is similar to the ultrasound sensor 200, but the difference is in that a metamaterial layer 430 is used to replace the metamaterial layer 230. Similar to the metamaterial layer 230, the metamaterial layer 430 includes a flat portion 432 and a microstructure portion 434, in which the flat portion 432 is located between the microstructure portion 434 and the transducer layer 220, and has a flat surface 432a. The microstructure portion 434 is located on the surface 432a of the flat portion 432 and includes at least one intrusion structure 434a. The intrusion structure 434a is arranged on the surface 432a of the flat portion 432 in a spiral shape, as shown in FIG. 4.

As shown in FIG. 5, in this embodiment, a cross-sectional view of the intrusion structure 434a is in a taper shape, and the angle θ is included between the surface 432a of the flat portion 432 and the surface of the intrusion structure 434a. The intrusion structure 434a has the height H and is formed on the flat portion 432 to have the pitch P.

It can be understood from the above illustration that the metamaterial layer 430 is similar to the metamaterial layer 230, but the difference is in that the metamaterial layer 430 includes only one intrusion structure 434a, and the intrusion structure 434a is formed on the flat portion 432 to have the pitch P. Therefore, a focus function provided by the metamaterial layer 430 is similar to the focus function provided by the metamaterial layer 230.

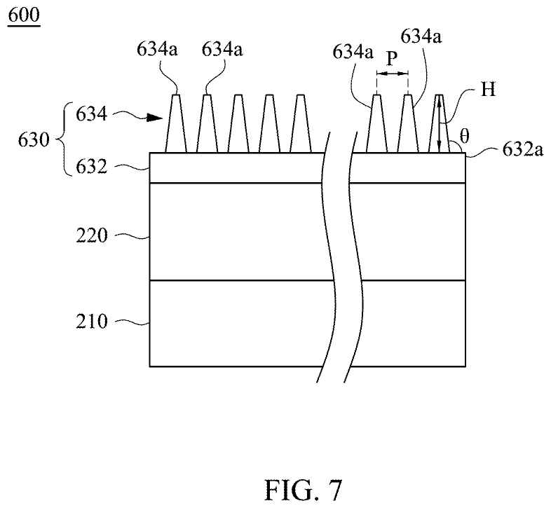

Referring to FIG. 6 and FIG. 7 simultaneously, FIG. 6 is a schematic diagram illustrating a top view of an ultrasound sensor 600 in accordance with embodiments of the present invention, and FIG. 7 is a schematic diagram illustrating a cross-sectional view of the ultrasound sensor 600 along a cut line C-C′. The ultrasound sensor 600 is similar to the ultrasound sensor 200, but the difference is in that a metamaterial layer 630 is used to replace the metamaterial layer 230. Similar to the metamaterial layer 230, the metamaterial layer 630 includes a flat portion 632 and a microstructure portion 634, in which the flat portion 632 is located between the microstructure portion 634 and the transducer layer 220, and has a flat surface 632a. The microstructure portion 634 is located on the surface 632a of the flat portion 632 and includes plural intrusion structures 634a having annular shapes. The intrusion structures 634a are arranged on the surface 632a of the flat portion 632 in a shape of concentric circles, as shown in FIG. 6.

As shown in FIG. 7, in this embodiment, a cross-sectional view of the intrusion structure 634a is in a taper shape, and the angle θ is included between the surface 632a of the flat portion 632 and the surface of the intrusion structure 634a. Two adjacent intrusion structures 634a are arranged on the flat portion 632 to have the pitch P, and each of the intrusion structures 634a has the height H.

It can be understood from the above illustration that the metamaterial layer 630 is similar to the metamaterial layer 230, but the difference is in that the intrusion structures 634a of the metamaterial layer 630 are arranged in a shape of concentric circles. Therefore, a focus function provided by the metamaterial layer 630 is similar to the focus function provided by the metamaterial layer 230. In addition, in the embodiments of the present invention, a number of turns of the intrusion structure(s) in the spiral shape or in the shape of concentric circles is equal to or greater than 1.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An ultrasound lens module configured to focus an ultrasound wave, wherein the ultrasound lens module comprises:
a metamaterial layer, wherein the metamaterial layer comprises:
a flat portion; and
a microstructure portion, located on a surface of the flat portion wherein the microstructure portion comprises at least one intrusion structure, wherein:
when a number of the at least one intrusion structure is one, the intrusion structure is disposed on the flat portion in a spiral shape;
when the number of the at least one intrusion structure is greater than one, the intrusion structures are disposed on the flat portion in spiral shapes or in a shape of concentric circles.

2. The ultrasound lens module of claim 1, wherein a pitch of the at least one intrusion structure is equal to a multiple of a half of a wavelength of the ultrasound wave or to a multiple of a quarter of the wavelength of the ultrasound wave.

3. The ultrasound lens module of claim 1, wherein a height of the at least one intrusion structure is equal to a multiple of a half of a wavelength of the ultrasound wave or to a multiple of a quarter of the wavelength of the ultrasound wave.

4. The ultrasound lens module of claim 1, wherein an angle included between the at least one intrusion structure and the surface of the flat portion is greater than 90 degrees and smaller than 180 degrees.

5. The ultrasound lens module of claim 1, wherein the material of the metamaterial layer is ceramic or polymethyl methacrylate (PMMA).

6. An ultrasound needle device comprising:
a transducer layer configured to emit an ultrasound wave; and
an ultrasound lens module disposed on the transducer layer to focus the ultrasound wave, wherein the ultrasound lens module comprises:
a metamaterial layer, wherein the metamaterial layer comprises:
a flat portion; and
a microstructure portion, located on a surface of the flat portion wherein the microstructure portion comprises at least one intrusion structure, wherein:
when a number of the at least one intrusion structure is one, the intrusion structure is disposed on the flat portion in a spiral shape;
when the number of the at least one intrusion structure is greater than one, the intrusion structures are disposed on the flat portion in spiral shapes or in a shape of concentric circles.

7. The ultrasound needle device of claim 6, wherein a pitch of the at least one intrusion structure is equal to a multiple of a half of a wavelength of the ultrasound wave or to a multiple of a quarter of the wavelength of the ultrasound wave.

8. The ultrasound needle device of claim 6, wherein a height of the at least one intrusion structure is equal to a multiple of a half of a wavelength of the ultrasound wave or to a multiple of a quarter of the wavelength of the ultrasound wave.

9. The ultrasound needle device of claim 6, wherein an angle included between the at least one intrusion structure and the surface of the flat portion is greater than 90 degrees and smaller than 180 degrees.

10. The ultrasound needle device of claim 6, wherein the material of the metamaterial layer is ceramic or polymethyl methacrylate (PMMA).
